(19) United States
(12) Patent Application Publication
(10) Pub. No.: US 2020/0038125 A1
(43) Pub. Date: Feb. 6, 2020

(54) ROBOTIC SURGICAL SYSTEMS, INSTRUMENTS, AND CONTROLS

(71) Applicant: Covidien LP, Mansfield, MA (US)

(72) Inventor: Jared Farlow, Los Angeles, CA (US)

(21) Appl. No.: 16/488,613

(22) PCT Filed: Mar. 14, 2018
(86) PCT No.: PCT/US2018/022288

§ 371 Date: Aug. 26, 2019

Related U.S. Application Data
(60) Provisional application No. 62/471,716, filed on Mar. 15, 2017.

(51) Int. Cl. A61B 34/37; A61B 34/00; G06F 3/0481; G06F 3/0484

(52) U.S. Cl. CPC A61B 34/37; A61B 34/25; A61B 2034/301; G06F 3/04817; G06F 3/04847; A61B 34/74

(57) ABSTRACT

A graphical user interface on a display of a user interface of a robotic surgical system includes a work area and a ribbon. The work area is configured to display a representation of a first end effector at a surgical site. The ribbon has a first icon that is configured to display end effector data of the first end effector.

### CROSS-REFERENCE TO RELATED APPLICATION

This application claims benefit of, and priority to, U.S. Provisional Patent Application Ser. No. 62/471,716, filed Mar. 15, 2017, the entire contents of which are hereby incorporated by reference.

### BACKGROUND

Robotic surgical systems have been used in minimally invasive medical procedures. During a medical procedure, the robotic surgical system is controlled by a surgeon interfacing with a user interface. The user interface allows the surgeon to manipulate an end effector of a surgical instrument that acts on a patient. The user interface includes an input controller or handle that is moveable by the surgeon to control the robotic surgical system.

Different robotic surgical systems exist in the market each with different controls and displays. As a surgeon moves from one robotic surgical system to another, the surgeon must familiarize themselves with the controls and the displays of the particular robotic surgical system. In addition, during a surgical procedure, each system may have different alerts or alarms to indicate a condition of the robotic surgical system or the patient which may delay recognition of the alarm to a surgeon not familiar with the particular robotic surgical system.

### SUMMARY

This disclosure relates generally to standardized controls and displays for robotic surgical systems.

In an aspect of the present disclosure, a graphical user interface on a display of a user interface of a robotic surgical system includes a work area and a ribbon. The work area is configured to display a representation of a first end effector at a surgical site. The ribbon has a first icon that is configured to display end effector data of the first end effector.

In aspects, the end effector data includes an identifier of the first end effector, a type of the first end effector, a selected function of the first end effector, and a state of the first end effector. The state of the first end effector may be indicative of a supply level of an exhaustible resource of the first end effector.

In some aspects, the ribbon includes an endoscope icon that is configured to display endoscope data of an endoscope providing a view of the surgical site. The endoscope data may include at least one of a rotation indicator or an inclination indicator. The first icon may be displayed on a left side of the endoscope icon when the first end effector is controlled by a left handle of the user interface and on a right side of the endoscope icon when the first end effector is controlled by a right handle of the user interface. The ribbon may include a second icon that is configured to display end effector data of a second end effector having a representation displayed in the work area. The second icon may be displayed on a left side of the endoscope icon when the second end effector is controlled by a left handle of the user interface and on a right side of the endoscope icon when the second end effector is controlled by a right handle of the user interface.

In certain aspects, the ribbon includes an endoscope control icon to indicate when an endoscope providing a view of the surgical site is being controlled by a user. The endoscope control icon may be displayed on a side of the ribbon indicative of a control handle of a surgical robot in control of the endoscope.

In some aspects, the ribbon includes a clutch icon to indicate when a control handle of the user interface is clutched. The clutch icon may be displayed on a side of the ribbon indicative of the control handle of the user interface that is clutched.

In particular aspects, the graphical user interface includes a firing indicator that is displayed along two edges forming a corner opposite the ribbon. The firing indicator may be displayed when the first end effector is being fired. The graphical user interface may include an off-screen indicator that is displayed along an edge of the display when the first end effector is outside of the working area shown on the display. The off-screen indicator may be positioned along an edge of the display indicative of a direction of the first end effector outside of the working area.

In another aspect of the present disclosure, a graphical user interface of a display of a user interface of a robotic surgical system includes a representation of a respective handle of the user interface, and a plurality of selection boxes. The representation has a plurality of control interfaces. Each selection box of the plurality of selection boxes having an indicator associating the selection box with one of the control interfaces. Each of the selection boxes of the plurality of selection boxes displaying text of a function associated with the respective control interface. The at least one of the selection boxes is selectable to modify a function associated with the respective control interface.

In another aspect of the present disclosure, a method of interfacing with a user interface to simulate or control a surgical robot includes visualizing a first icon in a ribbon of a graphical user interface on a display of the user interface and moving a first control handle of the user interface to move a first end effector within a work area of the display. The first icon provides end effector data of the first end effector associated with the first control handle. The first icon has a stat indicator that provides indicia that the first end effector is active when the first control handle is associated with the first control handle. The first icon has a mode indicator that provides indicia of a current mode of the first end effector.

In aspects, the method includes firing the first end effector. The display may indicate a firing indicator along two edges forming a corner opposite the ribbon while the first end effector is being fired to provide visual indicia of the firing of the first end effector.

In some aspects, moving the first control handle includes moving the first control handle such that the first end effector is moved outside of the work area of the display. The display may include an off-screen indicator along an edge of the display indicative of a direction of the first end effector.

In certain aspects, the method includes associating an endoscope with the first control handle to move an endoscope providing a view on the display. An endoscope control icon may appear in the ribbon on a side indicative of the first control handle while movement of the first control handle is associated with the endoscope.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic view of a robotic surgical system in accordance with the present disclosure;

FIG. 2 is a view of a display of the robotic surgical system of FIG. 1 having a graphical user interface (“GUI”);

FIGS. 3A-3C are views of exemplary icons of the GUI of FIG. 2;

FIG. 4 is a view of another icon of the GUI of FIG. 2;

FIG. 5 is a view of the display of the robotic surgical system of FIG. 1 with the GUI indicating that a right handle of the robotic surgical system of FIG. 1 is controlling an endoscope;

FIG. 6 is a table of exemplary icons that may be displayed by the GUI;

FIG. 7 is a view of the display of the robotic surgical system of FIG. 1 with the GUI indicating that an end effector associated with a left handle of the robotic surgical system of FIG. 1 is firing;

FIG. 8 is a view of the display of the robotic surgical system of FIG. 1 with the GUI indicating that an end effector associated with the left handle of the robotic surgical system of FIG. 1 positioned outside the view on the display;

FIG. 9 is a view of the display of the robotic surgical system of FIG. 1 during a customization mode illustrating another GUI; and

FIG. 10 is a view of one of the handles of the robotic surgical system of FIG. 1.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closer to the clinician and the term “distal” refers to the portion of the device or component thereof that is farther from the clinician.

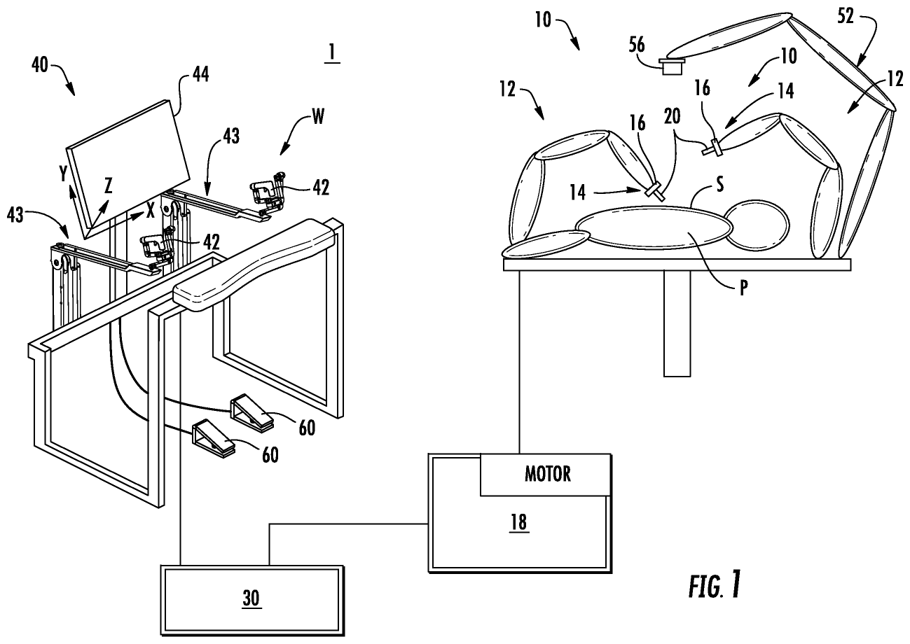

Referring to FIG. 1, a robotic surgical system 1 in accordance with the present disclosure is shown generally as a surgical robot 10, a processing unit 30, and a user console 40. The surgical robot 10 generally includes linkages 12 and a robot base 18. The linkages 12 moveably support an end effector or tool 20 which is configured to act on tissue. The linkages 12 may be in the form of arms each having an end 14 that supports the end effector or tool 20 which is configured to act on tissue. In addition, the ends 14 of the linkages 12 may include an imaging device 16 for imaging a surgical site “S”. The user console 40 is in communication with robot base 18 through the processing unit 30.

The user console 40 includes a display device 44 which is configured to display three-dimensional images. The display device 44 displays three-dimensional images of the surgical site “S” which may include data captured by imaging devices 16 positioned on the ends 14 of the linkages 12 and/or include data captured by imaging devices that are positioned about the surgical theater (e.g., an imaging device positioned within the surgical site “S”, an imaging device positioned adjacent the patient “P”, imaging device 56 positioned at a distal end of an imaging arm 52). The imaging devices (e.g., imaging devices 16, 56) may capture visual images, infra-red images, ultrasound images, X-ray images, thermal images, and/or any other known real-time images of the surgical site “S”. The imaging devices transmit captured imaging data to the processing unit 30 which creates three-dimensional images of the surgical site “S” in real-time from the imaging data and transmits the three-dimensional images to the display device 44 for display.

The user console 40 also includes input handles 42 which are supported on control arms 43 which allow a clinician to manipulate the surgical robot 10 (e.g., move the linkages 12, the ends 14 of the linkages 12, and/or the tools 20). Each of the input handles 42 is in communication with the processing unit 30 to transmit control signals thereto and to receive feedback signals therefrom. Additionally or alternatively, each of the input handles 42 may include input devices (not explicitly shown) which allow the surgeon to manipulate (e.g., clamp, grasp, fire, open, close, rotate, thrust, slice, etc.) the tools 20 supported at the ends 14 of the linkages 12.

Each of the input handles 42 is moveable through a predefined workspace to move the ends 14 of the linkages 12, e.g., tools 20, within a surgical site “S”. The three-dimensional images on the display device 44 are orientated such that the movement of the input handles 42 moves the ends 14 of the linkages 12 as viewed on the display device 44. The three-dimensional images remain stationary while movement of the input handles 42 is scaled to movement of the ends 14 of the linkages 12 within the three-dimensional images. To maintain an orientation of the three-dimensional images, kinematic mapping of the input handles 42 is based on a camera orientation relative to an orientation of the ends 14 of the linkages 12. The orientation of the three-dimensional images on the display device 44 may be mirrored or rotated relative to the view captured by the imaging devices 16, 56. In addition, the size of the three-dimensional images on the display device 44 may be scaled to be larger or smaller than the actual structures of the surgical site permitting a clinician to have a better view of structures within the surgical site “S”. As the input handles 42 are moved, the tools 20 are moved within the surgical site “S” as detailed below. Movement of the tools 20 may also include movement of the ends 14 of the linkages 12 which support the tools 20.

For a detailed discussion of the construction and operation of a robotic surgical system 1, reference may be made to U.S. Pat. No. 8,828,023, the entire contents of which are incorporated herein by reference.

The user console 40 further includes one or more foot pedals 60 that can be used to control various aspects of the robotic surgical system 1. For example, the foot pedal 60 may be selectively associated with an input handle, e.g., input handle 42, to actuate a tool 20 associated with the respective input handle. Additionally or alternatively, the foot pedal 60 may be associated with a camera, e.g., camera 56, to move the camera about the surgical site “S”. For a detailed discussion of suitable foot pedals, reference may be made to U.S. Provisional Patent Application Ser. No. 62/510,502, filed May 24, 2017, entitled “PEDAL CONTROL FOR ROBOTIC SURGICAL SYSTEMS,” and U.S. Provisional Patent Application Ser. No. 62/566,100, filed Sep. 8, 2017, entitled “HIGH PRECISION INSTRUMENT CONTROL MODE FOR ROBOTIC SURGICAL SYSTEMS,” the entire contents of each of the above applications are hereby incorporated by reference.

Referring now to FIG. 2, a graphical user interface (GUI) 100 of the display 44 is shown in accordance with the present disclosure. The GUI 100 includes a work area 110 and an information ribbon 120. The work area 110 displays images or representations of the surgical site S (FIG. 1). As shown, the work area 110 shows three surgical instruments each having an end effector or tool A, B, and C. The view of the work area 110 may be an image captured from one or more of the cameras, e.g., camera 56 (FIG. 1). Additionally or alternatively, the images in the work area 110 may include a representation of an endoscope E positioned within a surgical site which is providing the view of the work area 110 shown on the display 44.

The information ribbon 120 is presented in an area of the display 44 that minimizes obscuring of the work area 110 while providing a clinician information concerning the surgical instruments, end effectors, and/or endoscope viewable in the work area 110, e.g., end effectors A, B, and C and endoscope E. In addition, the information ribbon 120 may provide information to a clinician about surgical instruments, end effectors, and/or endoscopes of a surgical robot, e.g., surgical robot 10, that are outside of the work area 110. As shown the information ribbon 120 is located across the top of the display 44; however, it is contemplated that the information ribbon 120 may be located across the bottom of the display 44 and/or along a side of the display 44.

The information ribbon 120 includes icons displaying information relevant to the end effectors A, B, and C and the endoscope E. As shown, the information ribbon 120 includes an icon 122 which provides information with respect to the end effector A, an icon 124 which provides information with respect to the end effector B, an icon 126 which provides information with respect to the end effector C, and an icon 128 which provides information with respect to the endoscope E. The information ribbon 120 may have a defined border, e.g., a line, or may be borderless. The icons 122-128 are arranged across the information ribbon 120 with icons 122, 126 associated with end effectors manipulated by a left hand of the clinician, e.g., end effectors A and C, displayed on the left side of the information ribbon 120; icon 124 associated with end effectors manipulated by a right hand of the clinician, e.g., end effector B, displayed on the right side of the information ribbon 120; and the icon 128 which provides information with respect to the endoscope E is positioned between instruments controlled by the left hand and the right hand of the clinician and may be substantially centered in the information ribbon 120. The icons 122 and 126 are both associated with end effectors manipulated by the left hand of a clinician are arranged with the icon 126 to the left of the icon 122 to correspond to the position of the end effector C relative to the end effector A in the work area 110. The positions of the icons 122, 126 may switch or swap relative to one another as the positions of the end effectors A, C change during a surgical procedure. The positions may swap in real-time or there may be a slight delay such that the positions only swap if the relative positions of the end effectors A, C are incorrect, e.g., end effector C is positioned to the right of end effector A in the work area 110, for more than a predetermined amount of time, e.g., 5 seconds. This may reduce the number of swaps of icons 122, 126 which may distract a clinician during a surgical procedure.

The icons 122, 124, 126 provide information with respect to the end effector associated with the respective icon. The information is displayed in a standard format for each end effector but may differ based on the type of instrument of the end effector. For example, an icon for an electrosurgical instrument may have different information from an icon for a stapler or grasper.

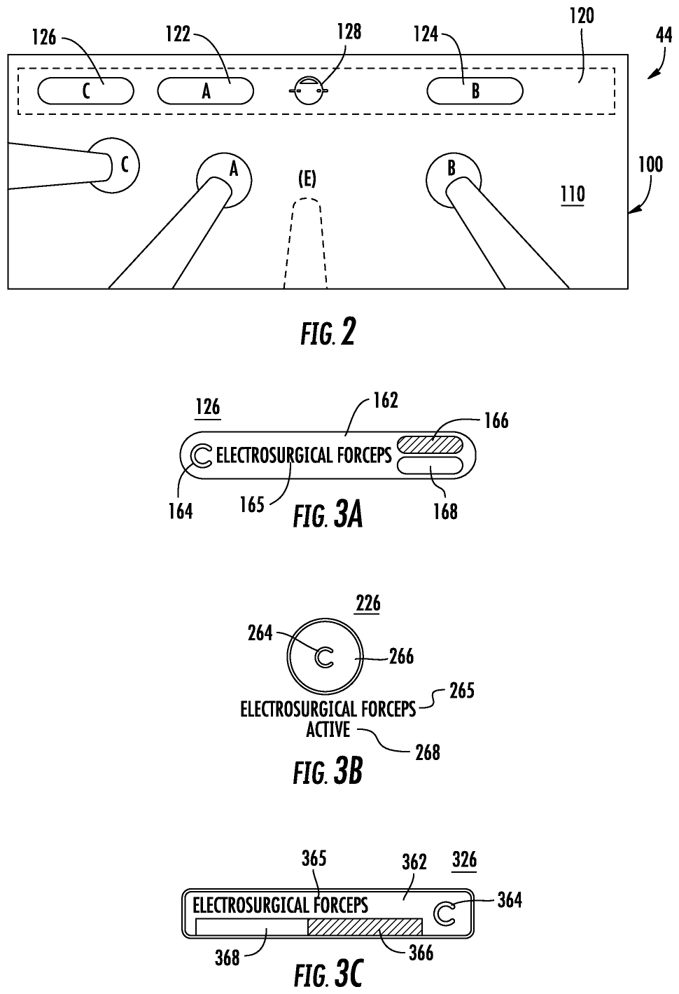

With particular reference to FIG. 3A, an embodiment of the icon 126 is provided to display information with respect to the associated end effector C. The icon 126 is substantially rectangular in shape with rounded ends and includes a background 162, an arm identifier 164, an instrument type indicator 165, a mode indicator 166, and a status indicator 168. The background 162 may be fixed or may indicate a status of the associated end effector. For example, when an end effector, e.g., end effector C, is connected to an arm, e.g., arm 12, of the surgical robot 10 (FIG. 1), the background 162 may have a first color, e.g., blue or cyan, and when no end effector is connected to the arm, the background 162 may be black. The arm identifier 164 displays information to identify which arm or link of a surgical robot that a particular icon is associated with. As shown, the arm identifier 164 displays a “C” to indicate that the icon 126 is displaying information related to arm C of the surgical robot 10 (FIG. 1).

The instrument type indicator 165 displays a type and/or name of an end effector or tool secured to the respective arm of the surgical robot 10 to allow a clinician to quickly identify a type of a tool secured to the respective arm. As shown, the instrument type 165 of the end effector C is an “Electrosurgical Forceps”.

The mode indicator 166 of the icon 126 provides information relevant to a selected mode of the associated end effector. As shown, the mode indicator 166 may be a first color, e.g., yellow, when the associated end effector is in a first mode, e.g., monopolar or MonoCUT, and may be a second color, e.g., green, when the associated end effector is in a second mode, e.g., bipolar or BiSEAL. When different types of end effectors are associated with the icon 126, the mode indicator 166 may display different modes. For example, when a stapling end effector is associated with the icon 126, the first mode may be indicative of a grasping mode and the second mode may be indicative of a stapling mode.

The status indicator 168 provides information relevant to a status of the associated end effector. For example, the status indicator 168 may display a first color, e.g., blue or cyan, when the associated end effector is active, e.g., under the control of a handle 42 of the user interface 40 (FIG. 1), and display a second color, e.g., red or yellow, when the associated end effector is not active, e.g., not under the control of a handle 42 of the user interface 40. Alternatively or additionally, the status indicator 168 may provide an indication of when a function of the associated end effector is being performed. For example, the status indictor 168 may display a third color, e.g., green, when a function, e.g., delivering electrosurgical energy or stapling, is being performed. In addition, the status indicator 168 may provide information indicative of a supply level of an exhaustible resource of the associated end effector. For example, when the associated end effector is a clip applier, the status indicator 168 may have a first size or length when the clip applier is full of clips and may reduce its size or length as each clip is fired from the clip applier.

With reference to FIG. 3B, another icon 226 is disclosed in accordance with the present disclosure. The icon 226 includes an arm identifier 264, an instrument type 265, a mode indicator 266, and a status indicator 268 the function of each of which is similar to the function of the like elements of the icon 126 detailed above and will not be discussed for reasons of brevity. The icon 226 does not include a defined background to increase the amount of the work area 110 of the display 44. As shown, the mode indicator 266 is circular in shape and includes the arm identifier 264 therein. The mode indicator 266 may have a variety of shapes. The shape of the mode indicator 266 may represent additional information related to the associated end effector, e.g., the type of end effector or the mode of the end effector. The instrument type 265 is displayed under the mode indicator 266. The status indicator 268 is displayed under the instrument type 265. In some embodiments, the shape of the mode indicator 266 represents the mode of the associated end effector and the color of the mode indicator 266 is indicative of the status of the associated end effector such that that status indicator 268 may be eliminated.

With reference to FIG. 3C, another icon 326 is disclosed in accordance with the present disclosure. The icon 326 includes a background 362, an arm identifier 364, an instrument type 365, a mode indicator 366, and a status indicator 368 the function of each of which is similar to the function of the like elements of the icon 126 detailed above and will not be discussed for reasons of brevity. The icon 326 is substantially rectangular in shape. The arm identifier 364 is disposed on the right of the background with the instrument type 365 displayed within the background 362 above the mode and status indicators 366, 368 which are displayed next to each other.

It will be appreciated that each of the icons 122, 124, 126 may have a style similar to the icons 126, 226, and 326 as detailed above. The icons 122, 124, 126 may have the same style or may each have a different style depending on the type of end effector associated with the respective icon. The information of each of the icons 122, 124, 126 may increase a clinician's situational awareness. Increasing a clinician's situational awareness may reduce duration of a procedure and/or improve a patient's outcome.

Referring back to FIG. 4, the icon 128 is a representation of an associated endoscope, e.g., endoscope E, and provides information with respect to the associated endoscope. The endoscope may be providing the view of the surgical site on the display 44. The icon 128 includes a rotation indicator 172 that indicates a degree of rotation of the associated endoscope about a longitudinal axis thereof from a neutral position (FIG. 2). As the associated endoscope is rotated about its longitudinal axis, the rotation indicator 172 rotates about the center of the icon 128. The icon 128 also includes an inclination indicator 174 that indicates the degree of inclination of the longitudinal axis of the associated endoscope. The inclination indicator 174 is a horizontal line with the interior of the icon 128 being filled below the horizontal line and being open above the horizontal line. When the inclination indicator 174 fills half of the interior of the icon 128, the associated endoscope is in a neutral position. The icon 128 may also increase a clinician's situational awareness.

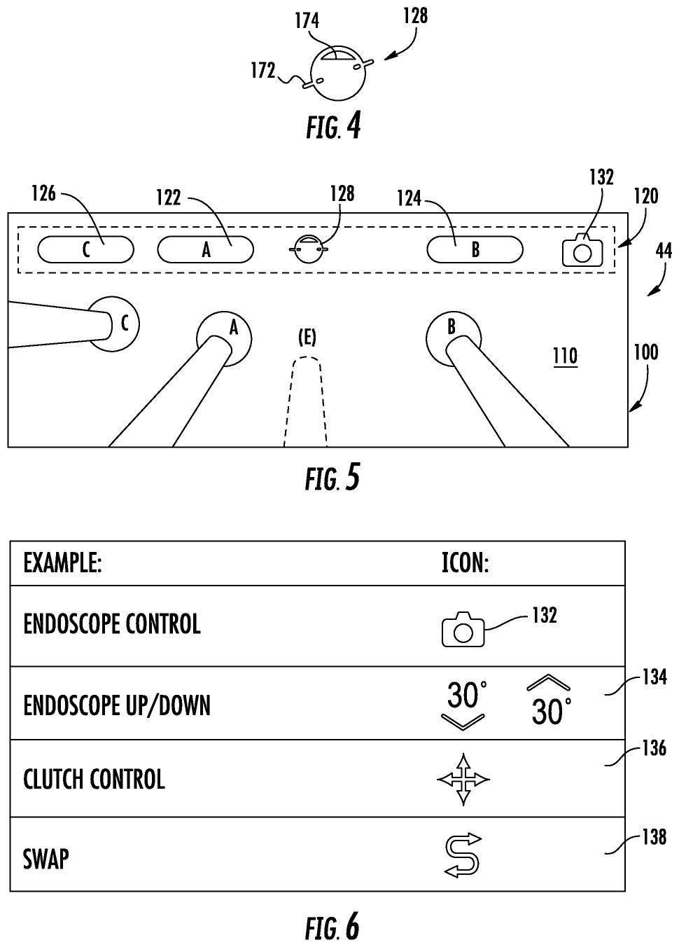

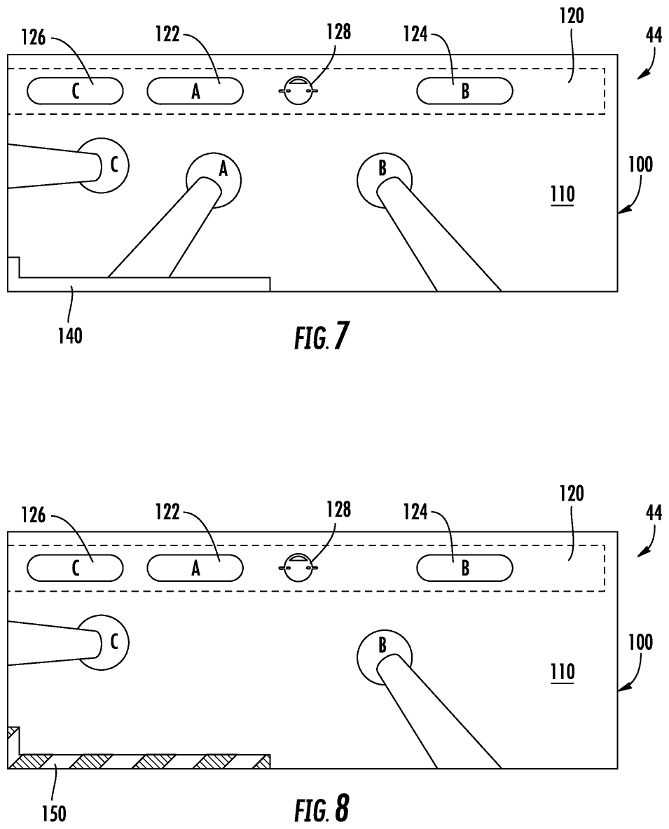

With reference to FIGS. 5 and 6, the information ribbon 120 may include additional icons to indicate other modes of control. For example, when a handle, e.g., handle 42 (FIG. 1), is being used to control an endoscope, e.g., endoscope E, an icon 132 is displayed in the information ribbon 120 on the respective side of the information ribbon 120. With particular reference to FIG. 5, the handle associated with the right hand of a clinician is being used for endoscope control. The information ribbon 120 may also display icon 134 when the inclination of an associated endoscope is being changed. When one of the handles is “clutched” the icon 136 may be displayed on the respective side to indicate that the respective handle, e.g., handle 42, is clutched. When control is being swapped from one end effector to another, e.g., from end effector A to end effector C, the icon 138 may be displayed on the respective side of the information ribbon 120. In addition, when control of an end effector is being swapped from one handle to another handle, e.g., from a left handle to a right handle, the icon 138 may be displayed to indicate that control of an end effector is being swapping sides, e.g., control of end effector A being swapped from the left handle to the right handle. It will be appreciated that when control is swapping sides that the icon associated with the swapping end effector may change sides of the information ribbon 120.

Referring now to FIG. 7, a firing indicator 140 of the GUI 100 is described in accordance with the present disclosure. The firing indicator 140 provides visual indicia to a clinician that an associated end effector is being fired. As shown in FIG. 7, the end effector A is active and being manipulated by a handle controlled by the left hand of the clinician. When the end effector A is being fired; e.g., delivering electrosurgical energy, firing a surgical fastener, applying a clip, and/or severing tissue with a knife; the firing indicator 140 is activated in a corner opposite the side of the GUI 100 having the information ribbon 120; e.g., the bottom of the GUI 100 when the information ribbon 120 is across the top of the GUI 100. For example, as shown, when the end effector A is being fired, the firing indicator 140 is positioned in the lower left corner of the GUI 100. The firing indicator 140 is maximized in size along a bottom edge and a side edge of the GUI 100 to increase the visibility of the firing indicator 140 without compromising a clinician's view of the working area 110. It will be appreciated that a distance that the firing indicator 140 extends away from the bottom edge and the side edge is minimized to reduce interference with the working area 110. The firing indicator 140 may be displayed as a solid color or may be displayed with a pattern. In addition, the firing indicator 140 may decrease in size as a predetermined duration of firing decreases to provide an indication of the duration of firing remaining. For example, the length of the firing indicator 140 across the bottom edge and/or the side edge may decrease during the firing. The firing indicator 140 may increase a clinician's situational awareness.

With reference to FIG. 8, an off-screen indicator 150 of the GUI 100 is described in accordance with the present disclosure. The off-screen indicator 150 provides visual indicia to a clinician that an associated end effector is outside of the working area 110. As shown in FIG. 8, the end effector A is outside of the working area 110 and being manipulated by a handle controlled by the left hand of the clinician. When the end effector A is outside of the working area 110, the off-screen indicator 150 is activated in a corner opposite the side of the GUI 100 having the information ribbon 120, e.g., the bottom of the GUI 100 when the information ribbon 120 is across the top of the GUI 100. For example, when the end effector A is outside of the working area 110, the off-screen indicator 150 is positioned in the lower left corner of the GUI 100. The off-screen indicator 150 is maximized in size along a bottom edge and a side edge of the GUI 100 to increase the visibility of the off-screen indicator 150 without compromising a clinician's view of the working area 110. Additionally or alternatively, the off-screen indicator 150 may be positioned on an edge of the GUI 100 to indicate the position of the end effector A outside of the working area 110. In such embodiments, the off-screen indicator 150 may move around edges of the GUI 100 as the end effector A is moved outside of the working area 110 and/or as the view of the working area 110 is moved. The off-screen indicator 150 may be displayed with a pattern of alternating colors. Alternatively, the off-screen indicator 150 may be displayed as an arrow directed to the position of the end effector A outside of the working area 110. The off-screen indicator 150 may increase a clinician's situational awareness.

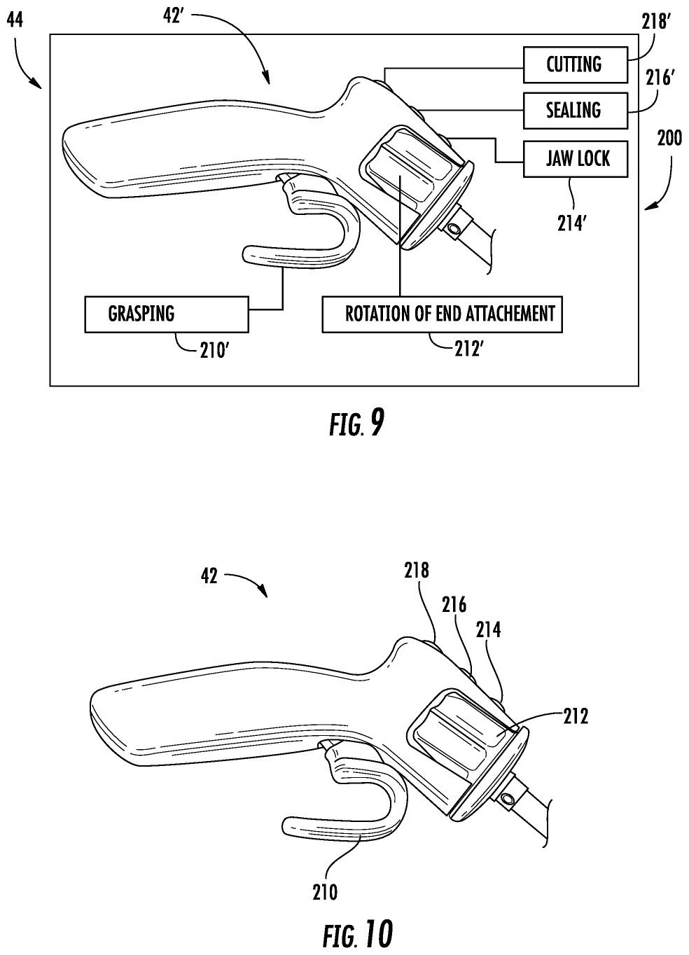

Referring now to FIGS. 9 and 10, a customization GUI 200 is provided in accordance with the present disclosure. The GUI 200 allows a clinician to understand and/or modify the controls of the handles 42. The GUI 200 includes a representation 42′ of the respective handle 42 with each control interface of the respective handle 42 identified with a corresponding function. For example, the label 210′ identifies the trigger 210 of the handle 42 and lists the corresponding function of the trigger 210 as “Grasping”. A clinician can use the GUI 200 to modify or map which control interface performs a particular function. For example, a clinician can use the GUI 200 to map function of “Jaw Lock” from the button 214 to the button 218 and to map the function of “Cutting” from the button 218 to the button 214 by modifying the selection in the boxes indicating the buttons 214 and 218. Further, a clinician can remove a function from one or more of the buttons, e.g., button 214, such that the respective button may be deactivated during a surgical procedure. It is contemplated that a clinician may create a profile that is assigned to the clinician such that each time the clinician signs into the profile, the clinician's customized settings are automatically assigned to the robotic surgical system 1 (FIG. 1). As detailed above, the GUI 200 may increase a clinician's situational awareness.

As detailed above and shown in FIG. 1, the user interface 40 is in operable communication with the robot system 10 to perform a surgical procedure on a patient “P”; however, it is envisioned that the user interface 40 may be in operable communication with a surgical simulator (not shown) to virtually actuate a robot system and/or tool in a simulated environment. For example, the surgical robot system 1 may have a first mode where the user interface 40 is coupled to actuate the robot system 10 and a second mode where the user interface 40 is coupled to the surgical simulator to virtually actuate a robot system. The surgical simulator may be a standalone unit or be integrated into the processing unit 30. The surgical simulator virtually responds to a clinician interfacing with the user interface 40 by providing visual, audible, force, and/or haptic feedback to a clinician through the user interface 40. For example, as a clinician interfaces with the input device handles 42, the surgical simulator moves representative tools that are virtually acting on tissue at a simulated surgical site. It is envisioned that the surgical simulator may allow a clinician to practice a surgical procedure before performing the surgical procedure on a patient. In addition, the surgical simulator may be used to train a clinician on a surgical procedure. Further, the surgical simulator may simulate “complications” during a proposed surgical procedure to permit a clinician to plan a surgical procedure.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A graphical user interface on a display of a user interface of a robotic surgical system, the graphical user interface comprising:
a work area configured to display a representation of a first end effector at a surgical site; and
a ribbon having a first icon configured to display end effector data of the first end effector.

2. The graphical user interface according to claim 1, wherein the end effector data includes an identifier of the first end effector, a type of the first end effector, a selected function of the first end effector, and a state of the first end effector.

3. The graphical user interface according to claim 2, wherein the state of the first end effector is indicative of a supply level of an exhaustible resource of the first end effector.

4. The graphical user interface according to claim 1, wherein the ribbon includes an endoscope icon configured to display endoscope data of an endoscope providing a view of the surgical site.

5. The graphical user interface according to claim 4, wherein the endoscope data includes at least one of a rotation indicator or an inclination indicator.

6. The graphical user interface according to claim 4, wherein the first icon is displayed on a left side of the endoscope icon when the first end effector is controlled by a left handle of the user interface and on a right side of the endoscope icon when the first end effector is controlled by a right handle of the user interface.

7. The graphical user interface according to claim 4, wherein the ribbon includes a second icon configured to display end effector data of a second end effector having a representation displayed in the work area.

8. The graphical user interface according to claim 7, wherein the second icon is displayed on a left side of the endoscope icon when the second end effector is controlled by a left handle of the user interface and on a right side of the endoscope icon when the second end effector is controlled by a right handle of the user interface.

9. The graphical user interface according to claim 1, wherein the ribbon includes an endoscope control icon to indicate when an endoscope providing a view of the surgical site is being controlled by a user.

10. The graphical user interface according to claim 9, wherein the endoscope control icon is displayed on a side of the ribbon indicative of a control handle of a surgical robot in control of the endoscope.

11. The graphical user interface according to claim 1, wherein the ribbon includes a clutch icon to indicate when a control handle of the user interface is clutched.

12. The graphical user interface according to claim 11, wherein the clutch icon is displayed on a side of the ribbon indicative of the control handle of the user interface that is clutched.

13. The graphical user interface according to claim 1, further comprising a firing indicator displayed along a two edges forming a corner opposite the ribbon, the firing indicator displayed when the first end effector is being fired.

14. The graphical user interface according to claim 1, further comprising an off-screen indicator displayed along an edge of the display when the first end effector is outside of the working area shown on the display.

15. The graphical user interface according to claim 14, wherein the off-screen indicator is positioned along an edge of the display indicative of a direction of the first end effector outside of the working area.

16. A graphical user interface on a display of a user interface of a robotic surgical system, the graphical user interface comprising:
a representation of a respective handle of the user interface, the representation having a plurality of control interfaces; and
a plurality of selection boxes, each selection box of the plurality of selection boxes having an indicator associating the selection box with one of the control interfaces, each of the selection boxes of the plurality of selection boxes displaying text of a function associated with the respective control interface, at least one of the selection boxes selectable to modify a function associated with the respective control interface.

17. A method of interfacing with a user interface to simulate or control a surgical robot, the method comprising:
visualizing a first icon in a ribbon of a graphical user interface on a display of the user interface, the first icon providing end effector data of a first end effector of a surgical robot associated with a first control handle of the user interface, the first icon having a state indicator providing indicia that the first end effector is active when the first control handle is associated with the first control handle, the first icon having a mode indicator providing indicia of a current mode of the first end effector; and
moving the first control handle of the user interface to move the first end effector within a work area of the display.

18. The method according to claim 17, further comprising firing the first end effector, the display including a firing indicator along two edges forming a corner opposite the ribbon while the first end effector is being fired to provide visual indicia of the firing of the first end effector.

19. The method according to claim 17, wherein moving the first control handle includes moving the first control handle such that the first end effector is moved outside of the work area of the display, the display including an off-screen indicator along an edge of the display indicative of a direction of the first end effector.

20. The method according to claim 17, further comprising associating an endoscope with the first control handle to move an endoscope providing a view on the display, an endoscope control icon appearing in the ribbon on a side indicative of the first control handle while movement of the first control handle is associated with the endoscope.
